(19) United States
(12) Patent Application Publication
(10) Pub. No.: US 2010/0098762 A1
(43) Pub. Date: Apr. 22, 2010

(54) Thermosensitive Pluronic Derivative Hydrogels With High Biodegradability And Biocompatibility For Tissue Regeneration And Preparation Method Thereof

(76) Inventors: Dong Keun Han, Seoul (KR); Kwideok Park, Seoul (KR); Jae-Jin Kim, Seoul (KR)

Correspondence Address: JONES DAY, 222 EAST 41ST ST, NEW YORK, NY, 10017, US

(21) Appl. No.: 12/573,037

(22) Filed: Oct. 2, 2009

(30) Foreign Application Priority Data
Oct. 22, 2008 (KR) 1020080103809

(51) Int. Cl. A61K 9/10; A61K 38/18; A61K 38/06; A61K 38/07; A61K 38/08; A61P 19/04

(52) U.S. Cl. 424/486; 514/12; 514/18; 514/17; 514/15

(57) ABSTRACT

The present invention relates to a thermosensitive pluronic derivative hydrogel for tissue regeneration in which a biodegradable polymer is introduced at one end or both ends of a pluronic polymer, a methacryloxyethyl trimellitic acid anhydride is conjugated to the biodegradable polymer, and a physiologically active substance is fixed to the methacryloxyethyl trimellitic acid anhydride, as well as a method for the preparation thereof. The pluronic derivative hydrogel according to the present invention exhibits high biodegradability due to the introduction of a biodegradable polymer while still maintaining the themosensitivity of the pluronic polymer itself and shows good biocompatibility owing to the coupling with a physiologically active substance capable of improving cell adhesion, proliferation and differentiation. Therefore, the pluronic derivative hydrogel according to the present invention can be effectively used in the regeneration of various kinds of tissues and organs.

The present application claims priority from Korean Patent Application No. 10-2008-0103809 filed Oct. 22, 2008, the subject matter of which is incorporated herein by reference in its entirety.

### FIELD OF THE INVENTION

The present invention relates to a thermosensitive pluronic derivative hydrogel for tissue regeneration in which a biodegradable polymer(s) and a physiologically active substance(s) are conjugated to a thermosensitive pluronic polymer by using a methacryloxyethyl trimellitic acid anhydride as a linker; and a method for the preparation thereof.

### BACKGROUND OF THE INVENTION

Tissue engineering is a new field that has been developed with the progress of science and that involves concepts and techniques from various fields of sciences, such as life science, engineering, medical science, and the like. Tissue engineering aims to understand the relationship between the structure and function of body tissues and to produce a biological substitute for damaged body tissues or organs for transplantation purposes so as to maintain, improve or restore the function of human body.

Tissue engineering techniques using hydrogels can be largely divided into two categories. In one technique, a target tissue is removed from a patient body and cells are isolated from the removed tissue. When the isolated cells are cultured to allow sufficient proliferation, these cells were then mixed with an injectable hydrogel scaffold and directly injected into the human body. Alternatively, the isolated cells are seeded on an injectable hydrogel scaffold, cultured in vitro for a predetermined period, and injected into the human body. In this technique, the injected hydrogel scaffold in a sol-state is converted into a gel in vivo at body temperature. Further, as oxygen and nutrients are provided to the transplanted cells in the hydrogel scaffold due to the diffusion of body fluids, blood vessels are newly formed within the hydrogel scaffold. When the blood vessels are formed and blood is provided to the cells, the cells proliferate and differentiate, forming new tissues and organs, while the hydrogel scaffold is degraded and eventually disappears.

In the other technique, an injectable hydrogel and a specific drug are mixed, and the resulting mixture is directly injected into the human body. As the injected hydrogel is converted to a gel at body temperature and is gradually degraded, the drug is released with a proper concentration for a prolonged period of time.

For studying tissue engineering, it is important to develop thermosensitive hydrogels that are similar to human tissue and are capable of being converted to a gel near body temperature. The hydrogel used for tissue regeneration should be thermosensitive so that it can be converted into a gel around body temperature while being maintained as a sol at room temperature. Further, the hydrogel should have good cell compatibility so that the tissue cells form a new tissue having a three-dimensional structure within the hydrogel. It should also be capable of serving as a barrier between the transplanted cells and the host cells.

Representative polymer hydrogels having such a unique thermosensitivity may include pluronic (P. Holmqvist et al., Int. J. Pharm. 194: 103, 2000), polyNIPAM (M. Harmon et al., Macromolecules 36: 1, 2003), hyaluronic acid (HA)(M. Ogiso et al., J. Biomed. Mater. Res. 39: 3, 1998), linear polyethylene glycol (PEG)-poly(lactic-co-glycolic acid) copolymer (PLGA)-polyethylene glycol (PEG)(B. Jeong et al., J. Biomed. Mater. Res. 50: 2, 2000), linear polyethylene glycol (PEG)-polylactic acid (PLA)-polyethylene glycol (PEG), star-shaped polylactic acid (PLA)-polyethylene glycol (PEG), star-shaped poly-ε-caprolactone (PCL)-polyethylene glycol (PEG)(S. Zhao et al., J. Func. Polym. 15: 1, 2002) and the like. Among them, it has been found that polyNIPAM has its own toxicity, while the other hydrogels do not have sufficient mechanical properties and biocompatibility to be used for tissue regeneration. Only hyaluronic acid and some pluronic derivatives were approved by the U.S. Food & Drug Administration (FDA) as injectable polymers which may be used in the human body.

Pluronic polymers are exemplified by the F series (beginning with “F”) of F38, F68, F77, F77, F98, F108, F127 derivatives, L series (beginning with “L”) of L31, L42, L43, L44, L62, L72, L101 derivatives, and P series (beginning with “P”) of P75, P103, P104 derivatives, all of them being trademarks. The pluronic polymers all have a structure of polyethylene oxide (PEO)-polypropylene oxide (PPO)-polyethylene oxide (PEO) with the only difference in their composition ratios of PEO:PPO:PEO and morphology. In particular, a pluronic F68 polymer having a molecular weight of about 8,700 daltons and a pluronic F127 polymer having a molecular weight of about 12,600 daltons, which were approved by the FDA, have been widely used as a biomaterial.

However, as the pluronic polymer increasingly becomes a macromolecule in order to meet the demand of high functionality, there are problems in that there are side effects resulting from the incomplete degradation and remaining residues in vivo. Therefore, the present inventors have developed pluronic derivative hydrogels with high biodegradability and biocompatibility which can be effectively used for tissue regeneration without causing any side effects.

### SUMMARY OF THE INVENTION

One of the objectives of the present invention is to provide a thermosensitive pluronic derivative hydrogel for tissue regeneration which shows high biocompatibility and biodegradability while maintaining the thermosensitivity of the pluronic polymer itself.

In order to achieve the above objective, one embodiment of the present invention relates to a thermosensitive pluronic derivative hydrogel for tissue regeneration having a structure in which a biodegradable polymer is introduced at one end or both ends of a thermosensitive pluronic polymer, methacryloxyethyl trimellitic acid anhydride is conjugated to the biodegradable polymer, and a physiologically active substance is fixed to the methacryloxyethyl trimellitic acid anhydride.

Another embodiment of the present invention relates to a method of preparing the above injectable thermosensitive pluronic derivative hydrogel.

### DETAILED DESCRIPTION OF THE INVENTION

The thermosensitive pluronic derivative hydrogel according to the present invention is characterized in that it shows improved biodegradability where it is capable of being completely degraded in vivo after a certain period of time due to the introduction of a biodegradable polymer while still maintaining the thermosensitivity of the pluronic polymer itself, and exhibits high biocompatibility owing to the coupling with a physiologically active substance capable of improving cell adhesion, proliferation or differentiation.

The pluronic derivative hydrogel according to the present invention is a complex of a thermosensitive pluronic polymer, a biodegradable polymer, a methacryloxyethyl trimellitic acid anhydride and a physiologically active substance, and has a structure in which the biodegradable polymer is first introduced at one end or both ends of the pluronic polymer, the methacryloxyethyl trimellitic acid anhydride carrying a polymerizable double bond and a carboxyl group is conjugated to the biodegradable polymer, and the physiologically active substance capable of improving biocompatibility is then fixed to the carboxyl group of the methacryloxyethyl trimellitic acid anhydride used as a linker.

The thermosensitive pluronic polymers suitable for the present invention can be of any type, so long as they have a structure of polyethylene oxide (PEO)-polypropylene oxide (PPO)-polyethylene oxide (PEO), and may include the F series of F38, F68, F77, F77, F98, F108, F127 derivatives, L series of L31, L42, L43, L44, L62, L72, L101 derivatives, and P series of P75, P103, P104 derivatives (all of them being trademarks), but are not limited thereto. Among them, it is desirable to use a pluronic F68 polymer having a molecular weight of about 8,700 daltons and a pluronic F 127 polymer having a molecular weight of about 12,600 daltons that have been approved by the FDA. In one embodiment of the present invention, F127 (PEO:PPO:PEO=98:68:98) is used as a thermosensitive pluronic polymer.

As a biodegradable polymer capable of being introduced into the pluronic polymer according to the present invention, any nontoxic polymer may be used for the present invention, so long as it is capable of being degraded in a living body. Suitable examples of the biodegradable polymer include, but are not limited to, glycolide, lactide, ε-caprolactone, dioxanone, trimethylenecarbonate, anhydrides, orthoester, hydroxyalkanoate, phosphagene, amino acids and copolymers thereof. There is no limitation on the molecular weight of the biodegradable polymer used, but, for example, a biodegradable polymer having a weight average molecular weight (M_{w}) ranging from 50 to 10,000 daltons, specifically 100 to 5,000 daltons may be used.

When the biodegradable polymer is introduced at both ends of the pluronic polymer, each biodegradable polymer introduced into both ends may be the same or different.

In the pluronic derivative hydrogel according to the present invention, the methacryloxyethyl trimellitic acid anhydride is used as a linker for fixing the physiologically active substance to the pluronic derivative having the biodegradable polymer(s) introduced at its one or both ends, and can be derived from 4-methacryloxyethyl trimellitic acid (4-META) or 2-methacryloxyethyl trimellitic acid (2-META). The methacryloxyethyl trimellitic acid anhydride suitable for the present invention is characterized by containing a polymerizable double bond capable of interacting with the biodegradable polymer and a carboxyl group capable of interacting with the physiologically active substance.

As a physiologically active substance suitable for the pluronic derivative hydrogel according to the present invention, any substance may be used for the present invention, so long as it is capable of inducing tissue regeneration and has high in vivo differentiation potential and biocompatibility. The physiologically active substance can be introduced into the pluronic derivative through the interaction between its amino group and the carboxyl group of the methacryloxyethyl trimellitic acid anhydride. Suitable examples thereof include, but are not limited to, biocompatible ligand peptides and growth factors. Where the methacryloxyethyl trimellitic acid anhydride is conjugated to each biodegradable polymer introduced at both ends of the pluronic polymer, the physiologically active substances conjugated to two carboxyl groups of the methacryloxyethyl trimellitic acid anhydride may be the same or different.

Suitable examples of such a biocompatible ligand peptide include Arg-Gly-Asp (RGD), Arg-Glu-Asp-Val (REDV), Leu-Asp-Val (LDV), Tyr-Ile-Gly-Ser-Arg (YIGSR), Pro-Asp-Ser-Gly-Arg (PDSGR), Ile-Lys-Val-Ala-Val (IKVAV), Arg-Asn-Ile-Ala-Glu-Ile-Ile-Lys-Asp-Ala (RNIAEIIKDA) and the like, but are not limited thereto. RGD and PDSGR improve cell adhesiveness to all kinds of cell types, while REDV and LDV promote the proliferation of vascular endothelial cells. YIGSR promotes the proliferation of vascular cells, while IKVAV and RNIAEIIKDA promote that of nerve cells.

Further, suitable examples of such growth factors include transforming growth factor-β (TGF), insulin-like growth factor (IGF), epidermal growth factor (EGF), nerve growth factor (NGF), vascular endothelial growth factor (VEGF), fibroblast growth factor (FGF), hepatocyte growth factor (HGF), platelet-derived growth factor (PDGF), bone morphogenetic protein (BMP) and the like, but are not limited thereto.

In some embodiments, the thermosensitive pluronic derivative hydrogel for tissue regeneration according to the present invention in which the biodegradable polymers and physiologically active substances are conjugated to both ends of the pluronic polymer by using the 4-methacryloxyethyl trimellitic acid anhydride as a linker is represented by the following Formula I.

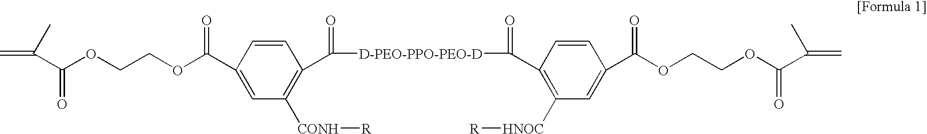

wherein -PEO-PPO-PEO- represents a pluronic polymer, D represents a biodegradable polymer, and R represents a physiologically active substance.

Further, the present invention provides a method of preparing the thermosensitive pluronic derivative hydrogel for tissue regeneration as described above.

The method according to the present invention comprises:

1) reacting a thermosensitive pluronic polymer with a biodegradable polymer, to thereby form a pluronic-biodegradable polymer hydrogel in which the biodegradable polymer is introduced at one end or both ends of the pluronic polymer;
2) reacting the pluronic-biodegradable polymer hydrogel formed in step 1) with a methacryloxyethyl trimellitic acid anhydride, to thereby form a pluronic-biodegradable polymer-methacryloxyethyl trimellitic acid anhydride hydrogel in which the methacryloxyethyl trimellitic acid anhydride is conjugated to the biodegradable polymer; and
3) reacting the pluronic-biodegradable polymer-methacryloxyethyl trimellitic acid anhydride hydrogel formed in step 2) with a physiologically active substance, to thereby form a pluronic-biodegradable polymer-methacryloxyethyl trimellitic acid anhydride-physiologically active substance hydrogel in which the physiologically active substance is fused to the methacryloxyethyl trimellitic acid anhydride.

Step 1) above is for preparing a pluronic-biodegradable polymer hydrogel by reacting a thermosensitive pluronic polymer with a biodegradable polymer, thereby introducing the biodegradable polymer at one end or both ends of the pluronic polymer. The reaction of step 1) is carried out by mixing the pluronic polymer and biodegradable polymer in a molar ratio ranging from 1:1 to 1:50, dissolving the resulting mixture in a solvent, and reacting the resulting solution at a temperature ranging from room temperature to 200° C. for 1 to 24 hours under a nitrogen atmosphere. Suitable solvents for the above reaction may include toluene, acetone, chloroform, dichloromethane, carbon tetrachloride, dioxan, tetrahydrofuran, and mixtures thereof. The reaction of step 1) can be carried out in the absence of a solvent.

The thermosensitive pluronic polymers suitable for this step have a structure of polyethylene oxide (PEO)-polypropylene oxide (PPO)-polyethylene oxide (PEO), and may include the F series of F38, F68, F77, F77, F98, F108, F127 derivatives, L series of L31, L42, L43, L44, L62, L72, L101 derivatives, and P series of P75, P103, P104 derivatives, but are not limited thereto. It is desirable to use a pluronic F68 polymer having a molecular weight of about 8,700 daltons or a pluronic F127 polymer having a molecular weight of about 12,600 daltons.

Suitable biodegradable polymers for this step may include, but are not limited to, glycolide, lactide, ε-caprolactone, dioxanon, trimethylenecarbonate, anhydrides, orthoester, hydroxyalkanoate, phosphagene, amino acids, and copolymers thereof, and can be appropriately selected depending on the desired rate of biodegradation. It is desirable to use a biodegradable polymer having a weight average molecular weight ranging from 50 to 10,000 daltons, more specifically, 100 to 5,000 daltons.

Step 2) above is for preparing a pluronic-biodegradable polymer-methacryloxyethyl trimellitic acid anhydride hydrogel by reacting the pluronic-biodegradable polymer hydrogel formed in step 1) with a methacryloxyethyl trimellitic acid anhydride, thereby conjugating the methacryloxyethyl trimellitic acid anhydride to the biodegradable polymer introduced at one end or both ends of the pluronic polymer. The reaction of step 2) is carried out by mixing the pluronic-biodegradable polymer hydrogel formed in step 1) and methacryloxyethyl trimellitic acid anhydride in a molar ratio ranging from 1:1 to 1:10, dissolving the resulting mixture in a solvent, and reacting the resulting solution at room temperature for 1 to 24 hours under a nitrogen atmosphere. Suitable solvents for the above reaction may include toluene, acetone, chloroform, dichloromethane, carbon tetrachloride, dioxan, tetrahydrofuran, and mixtures thereof.

The methacryloxyethyl trimellitic acid anhydride used in step 2) is a nontoxic substance used as a dental adhesive and has relatively good mechanical properties. In particular, since the methacryloxyethyl trimellitic acid anhydride carries a polymerizable double bond at one end and a carboxyl group at the other end, it can be polymerized with the biodegradable polymer by using the double bond and coupled with the physiologically active substance through the formation of an amide bond by using the carboxyl group. Suitable methacryloxyethyl trimellitic acid anhydride for this step may be 4-methacryloxyethyl trimellitic acid (4-META) anhydride or 2-methacryloxyethyl trimellitic acid (2-META) anhydride.

Each reaction of steps 1) and 2) may be carried out in the presence of a catalyst. Suitable catalysts for the present invention may include, but are not limited to, pyridine, trimethylamine, benzyldimethylamine, trimethylammoniumchloride, benzyltrimethylammoniumbromide, benzyltrimethylammoniumiodode, triphenylphosphine, triphenylstibine, methyltriphenylstibine, chromium 2-ethyl hexanoate, chromium octanoate, tin octanoate, dibutyltin dilaurate, 2-ethylzinc hexanoate, zinc octanoate, zirconium octanoate, dimethylsulfide and diphenylsulfide. Specifically, the catalyst of step 1) is used in a molar ratio ranging from 1:0.001 to 1:2 to the pluronic polymer, and that of step 2) is used in a molar ratio ranging from 1:0.001 to 1:2 to the pluronic-biodegradable polymer hydrogel.

Step 3) above is for forming a pluronic-biodegradable polymer-methacryloxyethyl trimellitic acid anhydride-physiologically active substance hydrogel by reacting the pluronic-biodegradable polymer-methacryloxyethyl trimellitic acid anhydride hydrogel formed in step 2) with a physiologically active substance, thereby fixing the physiologically active substance to a carboxyl group of the methacryloxyethyl trimellitic acid anhydride. The reaction of step 3) is carried out by mixing the pluronic-biodegradable polymer-methacryloxyethyl trimellitic acid anhydride hydrogel and physiologically active substance in a molar ratio ranging from 1:1 to 1:10, adding a catalyst thereto, and reacting the resulting mixture at room temperature for 1 to 24 hours.

Suitable catalysts for the reaction of step 3) may include 1-ethyl-3-(3-dimethylamino-propyl)carbodidimide (EDC), 1 1-cyclohexyl-3(2-morpholinoethyl) carbodiimide (CMC), dicyclohexyl carbodiimide (DCC), diisopropylcarbodiimide (DIC), N-ethyl-3-phenylisoxazolium-3′-sulfonate, N,N′-carbonyldiimidazole (CDI) and the like, but are not limited thereto. It is desirable to use EDC or CMC as a catalyst for facilitating the formation of the amide bond between the carboxyl group (—COOH) of the methacryloxyethyl trimellitic acid anhydride and an amine group (—NH₂) of the physiologically active substance. Here, the catalyst is used in a molar ratio ranging from 1:0.1 to 1:30 to the pluronic-biodegradable polymer-methacryloxyethyl trimellitic acid anhydride hydrogel.

As a physiologically active substance suitable for this step, any substance may be used for the present invention, so long as it is capable of providing biocompatibility to the pluronic derivative hydrogel. The physiologically active substance suitable for the present invention may be a biocompatible ligand peptide or a growth factor.

Suitable examples of such a biocompatible ligand peptide may include Arg-Gly-Asp (RGD), Arg-Glu-Asp-Val (REDV), Leu-Asp-Val (LDV), Tyr-Ile-Gly-Ser-Arg (YIGSR), Pro-Asp-Ser-Gly-Arg (PDSGR), Ile-Lys-Val-Ala-Val (IKVAV), Arg-Asn-Ile-Ala-Glu-Ile-Ile-Lys-Asp-Ala (RNIAEIIKDA) and the like, but are not limited thereto. Further, suitable examples of such a growth factor may include transforming growth factor-13 (TGF-(3), insulin-like growth factor (IGF), epidermal growth factor (EGF), neuron growth factor (NGF), vascular endotherial growth factor (VEGF), fibroblast growth factor (FGF), hepatocyte growth factor (HGF), platelet-derived growth factor (PDGF), bone morphogenetic protein (BMP), growth differentiation factor (GDF) and the like, but are not limited thereto.

The method of preparing the thermosensitive pluronic derivative hydrogel for tissue regeneration in accordance with the present invention is schematically illustrated by the following Scheme 1 where -PEO-PPO-PEO- represents a pluronic F127 polymer, G represents glycolide as a biodegradable polymer, META represents 4-methacryloxyethyl trimellitic acid anhydride, and R represents a physiologically active substance.

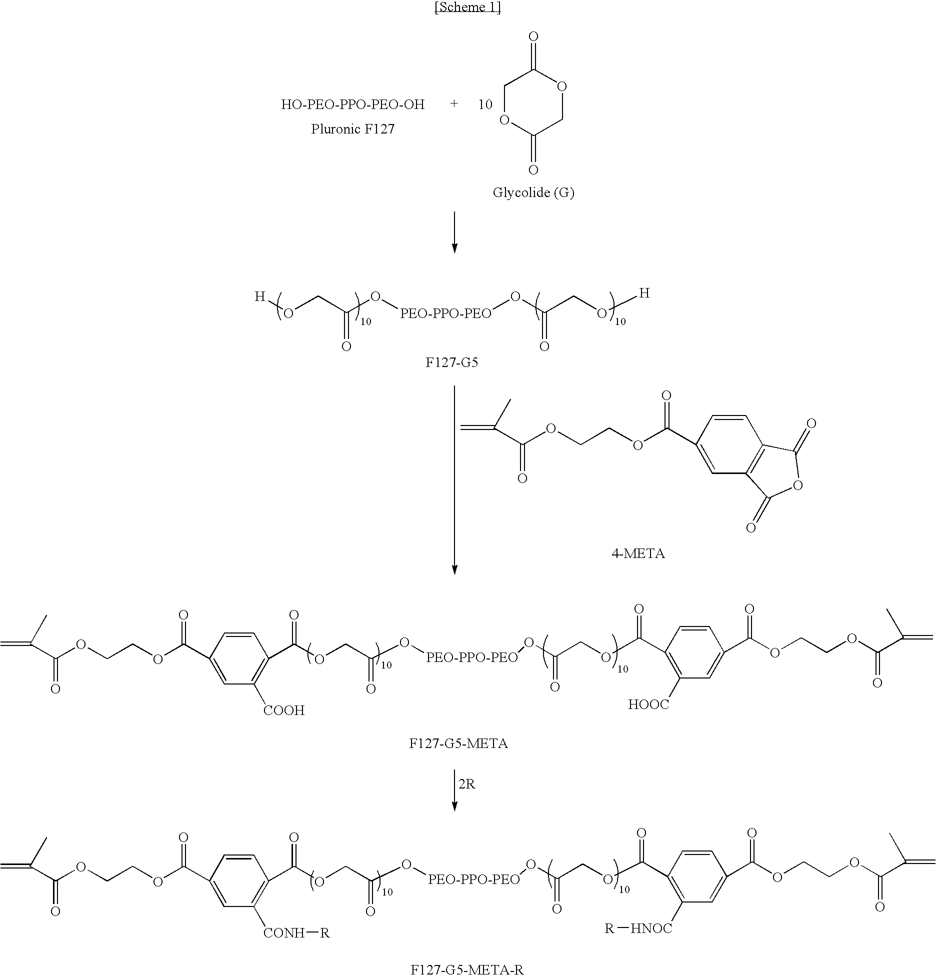

Referring to above Scheme 1, glycolide as a biodegradable polymer is introduced at both ends of a pluronic F127 polymer, to thereby form a pluronic F127-G5 hydrogel. Subsequently, 4-methacryloxyethyl trimellitic acid anhydride as a linker is conjugated to the glycolide of the pluronic F127-G5 hydrogel, thereby forming a pluronic F127-G5-META hydrogel. A physiologically active substance is then fixed to a carboxyl group of the 4-methacryloxyethyl trimellitic acid anhydride, which results in the formation of a pluronic F127-G5-META-R hydrogel.

As described above, the thermosensitive pluronic derivative hydrogel according to the present invention exhibits high biodegradability due to the introduction of a biodegradable polymer, while still maintaining the themosensitivity of the pluronic polymer itself, and shows good biocompatibility owing to the coupling with a physiologically active substance capable of improving cell adhesion, proliferation and differentiation. Therefore, the thermosensitive pluronic derivative hydrogel according to the present invention can be effectively used in the regeneration of various kinds of tissues and organs.

### EXAMPLES

Hereinafter, the embodiments of the present invention will be described in more detail with reference to the following examples. However, the examples are only provided for purposes of illustration and are not to be construed as limiting the scope of the invention.

### Example 1

### Preparation of Pluronic-Biodegradable Polymer Hydrogel

A pluronic F 127 polymer having a weight average molecular weight of about 12,600 daltons and glycolide (G) having a weight average molecular weight of about 116 daltons were mixed in a molar ratio of 1:10 and completely dissolved in 150 of toluene. The resulting mixture was then subjected to vacuum distillation to remove moisture, and then, its final volume was adjusted to 30 ml. After adding stannous octanoate as a catalyst in a molar ratio of 1:0.01 to the pluronic F127 polymer, the resulting mixture was reacted at 150° C. for 24 hours by stirring, followed by pouring the reaction mixture to 500 ml of cold ether to induce precipitation. A pluronic F127-G10 hydrogel as a precipitate was obtained in a high yield of >95%.

In order to examine whether the thus obtained pluronic F127-G10 hydrogel showed thermosensitivity, a sol-gel test according to a tube tiling method was performed at a temperature ranging from 15 to 90° C. As a result, it was found that although the pluronic F127-G10 hydrogel had a lower phase-transition temperature than the pluronic F127 polymer by approximately 1 to 2° C., it still maintained thermosensitivity.

### Example 2

### Preparation of Pluronic-Biodegradable Polymer-Methacryloxyethyl Trimellitic Acid Anhydride Hydrogel

A pluronic F127 polymer having a weight average molecular weight of about 12,600 daltons and lactide (L) having a weight average molecular weight of about 144 daltons were mixed in a molar ratio of 1:5 and completely dissolved in 150 ml of toluene. The resulting mixture was then subjected to vacuum distillation to remove moisture, and then, its final volume was adjusted to 30 ml. After adding stannous octanoate as a catalyst in a molar ratio of 1:0.01 to the pluronic F127 polymer, the resulting mixture was reacted at 150° C. for 24 hours by stirring, followed by pouring the reaction mixture to 500 ml, of cold ether to induce precipitation. A pluronic F127-L5 hydrogel as a precipitate was obtained in a high yield of >95%.

The thus obtained pluronic F127-L5 hydrogel was mixed with 4-META in a molar ratio of 1:2.2 and completely dissolved in 50 ml of toluene. Pyridine as a catalyst was added to the resulting mixture in a molar ratio of 1:0.01 to the pluronic F127-L5 hydrogel and reacted at room temperature for 24 hours by stirring. After the reaction was completed, the reaction mixture was poured to 500 ml of cold ether to induce precipitation, thereby obtaining a pluronic F127-L3-META hydrogel as a precipitate in a high yield of >90%.

As a result of examining its thermosensitivity through the sol-gel test as described in Example 1, it has been found that although the pluronic F127-L3-META hydrogel had a lower phase-transition temperature than the pluronic F127 polymer by approximately 2 to 3° C., it still maintained thermosensitivity.

### Example 3

### Preparation of Pluronic-Biodegradable Polymer-Methacryloxyethyl Trimellitic Acid Anhydride-Physiologically Active Substance Hydrogel

A pluronic F127 polymer having a weight average molecular weight of about 12,600 daltons and ε-caprolactone (C) having a weight average molecular weight of about 114 daltons were mixed in a molar ratio of 1:3 and completely dissolved in 150 ml of toluene. The resulting mixture was then subjected to vacuum distillation to remove moisture, and then, its final volume was adjusted to 30 ml. After adding stannous octanoate as a catalyst in a molar ratio of 1:1 to the pluronic F127 polymer, the resulting mixture was reacted at 150° C. for 24 hours by stirring, followed by pouring the reaction mixture to 500 ml of cold ether to induce precipitation. A pluronic F127-C3 hydrogel as a precipitate was obtained in a high yield of >95%.

The thus obtained pluronic F127-C3 hydrogel was mixed with 4-META in a molar ratio of 1:2.2 and completely dissolved in 50 ml of toluene. Pyridine as a catalyst was added to the resulting mixture in a molar ratio of 1:0.01 to the pluronic F127-C3 hydrogel and reacted at room temperature for 24 hours by stirring. After the reaction was completed, the reaction mixture was poured to 500 id of cold ether to induce precipitation, thereby obtaining a pluronic F127-C3-META hydrogel as a precipitate in a high yield of >90%.

The pluronic F127-C3-META hydrogel prepared above was completely dissolved in a 2-morpholino ethanesulfonic acid (MES) buffer in a weight ratio of 1:15, and then, EDC as a catalyst was added in a molar ratio of 1:20 to the pluronic F127-C3-META hydrogel to activate a carboxyl group of 4-META. After stirring for 2 hours, a biocompatible ligand peptide RGD as a physiologically active substance was added to the resulting mixture in a molar ratio of 1:2.1 to the pluronic F127-C3-META hydrogel and reacted at room temperature for 24 hours. After the reaction was completed, the reaction mixture was dialyzed using water for 48 hours and freeze-dried at −70° C. for 24 hours, to thereby obtain a pluronic F127-C3-META-RGD hydrogel in a high yield of >90%.

As a result of examining its thermosensitivity through the sol-gel test as described in Example 1, it was found that although the pluronic F127-C3-META-RGD hydrogel had a lower phase-transition temperature than the pluronic F127 polymer by approximately 4 to 5° C., it still maintained thermosensitivity.

Further, the result of investigating the effect of the pluronic F127-C3-META-RGD hydrogel at a concentration of 20% on the adhesion of chondrocytes showed that its cell adhesion activity was increased by approximately 90% as compared with the pluronic F127 polymer, suggesting an improvement in biocompatibility.

### Example 4

### Preparation of Pluronic-Biodegradable Polymer-Methacryloxyethyl Trimellitic Acid Anhydride-Physiologically Active Substance Hydrogel

The pluronic F127-G5L3-META-YIGSR hydrogel was prepared according to the same method as described in Example 3 (yield: >90%) except that a mixture of glycolide and lactide (mixing ratio=5:3) was used as a biodegradable polymer, a ligand peptide YIGSR relating to the proliferation of vascular cells was used as a physiologically active substance, and CMC was used as a catalyst.

As a result of examining its thermosensitivity through the sol-gel test as described in Example 1, it was found that although the pluronic F127-G5L3-META-YIGSR hydrogel had a lower phase-transition temperature than the pluronic F127 polymer by approximately 4 to 5° C., it still maintained thermosensitivity. Further, the result of investigating the effect of the pluronic F127-G5L3-META-YIGSR hydrogel at a concentration of 20% on the proliferation of vascular cells showed that its proliferation activity was increased by approximately 90% as compared with the pluronic F127 polymer, suggesting an improvement in biocompatibility.

### Example 5

### Preparation of Pluronic-Biodegradable Polymer-Methacryloxyethyl Trimellitic Acid Anhydride-Physiologically Active Substance Hydrogel

The pluronic F127-G5C1-META-IKVAV hydrogel was prepared according to the same method as described in Example 3 (yield: >90%) except that a mixture of glycolide and ε-caprolactone (mixing ratio=5:1) was used as a biodegradable polymer, and a ligand peptide IKVAV relating to the proliferation of nerve cells was used as a physiologically active substance.

As a result of examining its thermosensitivity through the sol-gel test as described in Example 1, it was found that although the pluronic F127-G5C1-META-IKVAV hydrogel had a lower phase-transition temperature than the pluronic F127 polymer by approximately 4 to 5° C., it still maintained thermosensitivity. Further, the result of investigating the effect of the pluronic F127-G5C1-META-IKVAV hydrogel at a concentration of 20% on the proliferation of nerve cells showed that its proliferation activity was increased by approximately 90% as compared with the pluronic F 127 polymer, suggesting an improvement in biocompatibility.

### Example 6

### Preparation of Pluronic-Biodegradable Polymer-Methacryloxyethyl Trimellitic Acid Anhydride-Physiologically Active Substance Hydrogel

The pluronic F127-L3C3-META-REDV hydrogel was prepared according to the same method as described in Example 3 (yield: >90%) except that a mixture of lactide and ε-caprolactone (mixing ratio=3:3) was used as a biodegradable polymer, and a ligand peptide REDV relating to the proliferation of vascular endothelial cells was used as a physiologically active substance.

As a result of examining its thermosensitivity through the sol-gel test as described in Example 1, it was found that although the pluronic F127-L3C3-META-REDV hydrogel had a lower phase-transition temperature than the pluronic F127 polymer by approximately 4 to 5° C., it still maintained thermosensitivity. Further, the result of investigating the effect of the pluronic F127-L3C3-META-REDV hydrogel at a concentration of 20% on the proliferation of vascular endothelial cells showed that its proliferation activity was increased by approximately 80% as compared with the pluronic F127 polymer, suggesting an improvement in biocompatibility.

### Example 7

### Preparation of Pluronic-Biodegradable Polymer-Methacryloxyethyl Trimellitic Acid Anhydride-Physiologically Active Substance Hydrogel

The pluronic F127-G5-META-TGF-β hydrogel was prepared according to the same method as described in Example 3 (yield: >90%) except that glycolide was used as a biodegradable polymer, and a growth factor TGF-β was used as a physiologically active substance.

As a result of examining its thermosensitivity through the sol-gel test as described in Example 1, it was found that although the pluronic F127-G5-META-TGF-β hydrogel had a lower phase-transition temperature than the pluronic F127 polymer by approximately 4 to 5° C., it still maintained thermosensitivity. Further, the result of investigating the effect of the pluronic F127-G5-META-TGF-β hydrogel at a concentration of 20% on the differentiation of chondrocytes showed that its differentiation activity was increased by approximately 80% as compared with the pluronic F127 polymer, suggesting an improvement in biocompatibility.

### Example 8

### Preparation of Pluronic-Biodegradable Polymer-Methacryloxyethyl Trimellitic Acid Anhydride-Physiologically Active Substance Hydrogel

The pluronic F127-L5-META-EGF hydrogel was prepared according to the same method as described in Example 3 (yield: >90%) except that lactide was used as a biodegradable polymer, a growth factor EGF was used as a physiologically active substance, and CMC was used as a catalyst.

As a result of examining its thermosensitivity through the sol-gel test as described in Example 1, it was found that although the pluronic F127-L5-META-EGF hydrogel had a lower phase-transition temperature than the pluronic F127 polymer by approximately 4 to 5° C., it still maintained thermosensitivity. Further, the result of investigating the effect of the pluronic F127-L5-META-EGF hydrogel at a concentration of 20% on the differentiation of cord blood stem cells into nerve cells showed that its differentiation activity was increased by approximately 80% as compared with the pluronic F127 polymer, suggesting an improvement in biocompatibility.

### Example 9

### Preparation of Pluronic-Biodegradable Polymer-Methacryloxyethyl Trimellitic Acid Anhydride-Physiologically Active Substance Hydrogel

The pluronic F127-C5-META-NGF hydrogel was prepared according to the same method as described in Example 3 (yield: >90%) except that the molar ratio of ε-caprolactone to the pluronic F127 polymer was 1:5, and a growth factor NGF was used as a physiologically active substance.

As a result of examining its thermosensitivity through the sol-gel test as described in Example 1, it was found that although the pluronic F127-05-META-NGF hydrogel had a lower phase-transition temperature than a pluronic F127 polymer by approximately 4 to 5° C., it still maintained thermosensitivity. Further, the result of investigating the effect of the pluronic F127-05-META-NGF hydrogel at a concentration of 20% on the differentiation of bone marrow stem cells into nerve cells showed that its differentiation activity was increased by approximately 90% as compared with the pluronic F127 polymer, suggesting an improvement in biocompatibility.

### Example 10

### Preparation of Pluronic-Biodegradable Polymer-Methacryloxyethyl Trimellitic Acid Anhydride-Physiologically Active Substance Hydrogel

The pluronic F127-G3-META-VEGF hydrogel was prepared according to the same method as described in Example 3 (yield: >90%) except that glycolide was used as a biodegradable polymer and a growth factor VEGF was used as a physiologically active substance.

As a result of examining its thermosensitivity through the sol-gel test as described in Example 1, it was found that although the pluronic F127-G3-META-VEGF hydrogel had a lower phase-transition temperature than the pluronic F127 polymer by approximately 4 to 5° C., it still maintained thermosensitivity. Further, the result of investigating the effect of the pluronic F127-G3-META-VEGF hydrogel at a concentration of 20% on the differentiation of embryonic stem cells into vascular endothelial cells showed that its differentiation activity was increased by approximately 80% as compared with the pluronic F127 polymer, suggesting an improvement in biocompatibility.

### Example 11

### Preparation of Pluronic-Biodegradable Polymer-Methacryloxyethyl Trimellitic Acid Anhydride-Physiologically Active Substance Hydrogel

The pluronic F127-L2-META-BMP-2 hydrogel was prepared according to the same method as described in Example 3 (yield: >90%) except that lactide was used as a biodegradable polymer and a growth factor BMP-2 was used as a physiologically active substance.

As a result of examining its thermosensitivity through the sol-gel test as described in Example 1, it was found that although the pluronic F127-L2-META-BMP-2 hydrogel had a lower phase-transition temperature than the pluronic F127 polymer by approximately 4 to 5° C., it still maintained thermosensitivity. Further, the result of investigating the effect of the pluronic F127-L2-META-BMP-2 hydrogel at a concentration of 20% on the differentiation of totipotent stem cells into osteocytes showed that its cell differentiation activity was increased by approximately 80% as compared with the pluronic F127 polymer, suggesting an improvement in biocompatibility.

As can be seen in Examples 3 to 11, the thermosensitive pluronic derivative hydrogels according to the present invention where the biodegradable polymer(s) and physiologically active substance(s) are introduced into the pluronic polymer by using the methacryloxyethyl trimellitic acid anhydride as a linker showed improved biocompatability in terms of cell adhesion, proliferation and differentiation while maintaining thermosensitivity of the pluronic polymer itself.

While the present invention has been described and illustrated with respect to a number of embodiments of the invention, it will be apparent to those skilled in the art that variations and modifications are possible without deviating from the broad principles and teachings of the present invention, which is defined by the claims appended hereto.

What is claimed:

1. A thermosensitive pluronic derivative hydrogel in which a biodegradable polymer is introduced at one end or both ends of a pluronic polymer, a methacryloxyethyl trimellitic acid anhydride is conjugated to said biodegradable polymer, and a physiologically active substance is fixed to said methacryloxyethyl trimellitic acid anhydride.

2. The thermosensitive pluronic derivative hydrogel according to claim 1, wherein the pluronic polymer has a structure of polyethylene oxide (PEO)-polypropylene oxide (PPO)-polyethylene oxide (PEO).

3. The thermosensitive pluronic derivative hydrogel according to claim 1, wherein the pluronic polymer is a pluronic F68 polymer having a weight average molecular weight of 8,700 daltons or a pluronic F127 polymer having a weight average molecular weight of 12,600 daltons.

4. The thermosensitive pluronic derivative hydrogel according to claim 1, wherein the biodegradable polymer is selected from the group consisting of glycolide, lactide, ε-caprolactone, dioxanone, trimethylenecarbonate, anhydrides, orthoester, hydroxyalkanoate, phosphagene, amino acids and copolymers thereof.

5. The thermosensitive pluronic derivative hydrogel according to claim 1, wherein the biodegradable polymers introduced at both ends of the pluronic polymer are the same or different.

6. The thermosensitive pluronic derivative hydrogel according to claim 1, wherein the methacryloxyethyl trimellitic acid anhydride is 4-methacryloxyethyl trimellitic acid (4-META) anhydride or 2-methacryloxyethyl trimellitic acid (2-META) anhydride.

7. The thermosensitive pluronic derivative hydrogel according to claim 1, wherein the physiologically active substance is a biocompatible ligand peptide or a growth factor.

8. The thermosensitive pluronic derivative hydrogel according to claim 7, wherein the biocompatible ligand peptide is selected from the group consisting of Arg-Gly-Asp (RGD), Arg-Glu-Asp-Val (REDV), Leu-Asp-Val (LDV), Tyr-11e-Gly-Ser-Arg (YIGSR), Pro-Asp-Ser-Gly-Arg (PDSGR), Ile-Lys-Val-Ala-Val (IKVAV) and Arg-Asn-Ile-Ala-Glu-Ile-Ile-Lys-Asp-Ala (RNIAEIIKDA).

9. The thermosensitive pluronic derivative hydrogel according to claim 1, wherein the growth factor is selected from the group consisting of transforming growth factor-β (TGF-β), insulin-like growth factor (IGF), epidermal growth factor (EGF), nerve growth factor (NGF), vascular endothelial growth factor (VEGF), fibroblast growth factor (FGF), hepatocyte growth factor (HGF), platelet-derived growth factor(PDGF) and bone morphogenetic protein (BMP).

10. The thermosensitive pluronic derivative hydrogel according claim 1 having a structure represented by the following Formula I: 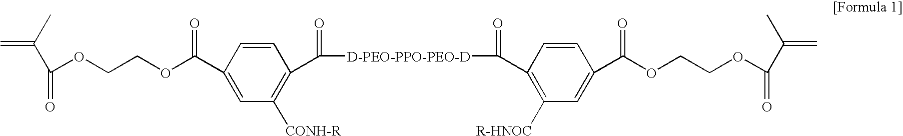
wherein, -PEO-PPO-PEO- represents a pluronic polymer, D represents a biodegradable polymer, and R represents a physiologically active substance.

11. A method of preparing a thermosensitive pluronic derivative hydrogel, comprising:
reacting a thermosensitive pluronic polymer with a biodegradable polymer, to thereby form a pluronic-biodegradable polymer hydrogel in which the biodegradable polymer is introduced at one end or both ends of the pluronic polymer;
reacting the pluronic-biodegradable polymer hydrogel with a methacryloxyethyl trimellitic acid anhydride, to thereby form a pluronic-biodegradable polymer-methacryloxyethyl trimellitic acid anhydride hydrogel in which the methacryloxyethyl trimellitic acid anhydride is conjugated to said biodegradable polymer; and
reacting the pluronic-biodegradable polymer-methacryloxyethyl trimellitic acid anhydride hydrogel with a physiologically active substance, to thereby form a pluronic-biodegradable polymer-methacryloxyethyl trimellitic acid anhydride-physiologically active substance hydrogel in which the physiologically active substance is fused to said methacryloxyethyl trimellitic acid anhydride.

12. The method according to claim 11, wherein the reacting a thermosensitive pluronic polymer is carried out by mixing the pluronic polymer and biodegradable polymer in a molar ratio ranging from 1:1 to 1:50, dissolving the resulting mixture in a solvent, and reacting the resulting solution at a temperature ranging from room temperature to 200° C. for 1 to 24 hours under a nitrogen atmosphere.

13. The method according to claim 12, wherein the solvent is selected from the group consisting of toluene, acetone, chloroform, dichloromethane, carbon tetrachloride, dioxan, tetrahydrofuran and mixtures thereof.

14. The method according to claim 11, wherein the thermosensitive pluronic polymer has a structure of polyethylene oxide (PEO)-polypropylene oxide (PPO)-polyethylene oxide (PEO).

15. The method according to claim 14, wherein the thermosensitive pluronic polymer is a pluronic F68 polymer having a weight average molecular weight of 8,700 daltons or a pluronic F127 polymer having a weight average molecular weight of 12,600 daltons.

16. The method according to claim 11, wherein the reacting the pluronic-biodegradable polymer hydrogel is carried out by mixing the pluronic-biodegradable polymer hydrogel and methacryloxyethyl trimellitic acid anhydride in a molar ratio ranging from 1:1 to 1:10, dissolving the resulting mixture in a solvent, and reacting the resulting solution at room temperature for 1 to 24 hours under a nitrogen atmosphere.

17. The method according to claim 16, wherein the solvent is selected from the group consisting of toluene, acetone, chloroform, dichloromethane, carbon tetrachloride, dioxan, tetrahydrofuran and mixtures thereof.

18. The method according to claim 11, wherein the methacryloxyethyl trimellitic acid anhydride is 4-methacryloxyethyl trimellitic acid (4-META) anhydride or 2-methacryloxyethyl trimellitic acid (2-META) anhydride.

19. The method according to claim 11, wherein each of the reacting a thermosensitive pluronic polymer and the reacting the pluronic-biodegradable polymer hydrogel is carried out in the presence of a catalyst.

20. The method according to claim 19, wherein the catalyst is selected from the group consisting of pyridine, trimethylamine, benzyldimethylamine, trimethylammoniumchloride, benzyltrimethylammoniumbromide, benzyltrimethylammoniumiodode, triphenylphosphine, triphenylstibine, methyltriphenylstibine, chromium 2-ethyl hexanoate, chromium octanoate, tin octanoate, dibutyltin dilaurate, 2-ethylzinc hexanoate, zinc octanoate, zirconium octanoate, dimethylsulfide and diphenylsulfide.

21. The method according to claim 19, wherein the catalyst is used in a molar ratio ranging from 1:0.001 to 1:2 to the pluronic polymer and the pluronic-biodegradable polymer hydrogel, respectively.

22. The method according to claim 11, wherein the reacting the pluronic-biodegradable polymer-methacryloxyethyl trimellitic acid anhydride hydrogel is carried out by mixing the pluronic-biodegradable polymer-methacryloxyethyl trimellitic acid anhydride hydrogel and physiologically active substance in a molar ratio ranging from 1:1 to 1:10, adding a catalyst thereto, and reacting the resulting mixture at room temperature for 1 to 24 hours.

23. The method according to claim 22, wherein the catalyst is selected from the group consisting of 1-ethyl-3-(3-dimethylamino-propyl)carbodidimide (EDC), 1-cyclohexyl-3-(2-morpholinoethyl) carbodiimide (CMC), dicyclohexyl carbodiimide (DCC), diisopropylcarbodiimide (DIC), N-ethyl-3-phenylisoxazolium-3′-sulfonate and N,N′-carbonyldiimidazole (CDI).

24. The method according to claim 22, wherein the catalyst is used in a molar ratio ranging from 1:0.1 to 1:30 to the pluronic-biodegradable polymer-methacryloxyethyl trimellitic acid anhydride hydrogel.

25. The method according to claim 11, wherein the physiologically active substance is a biocompatible ligand peptide or a growth factor.

26. The method according to claim 25, wherein the biocompatible ligand peptide is selected from the group consisting of Arg-Gly-Asp (RGD), Arg-Glu-Asp-Val (REDV), Leu-Asp-Val (LDV), Tyr-Ile-Gly-Ser-Arg (YIGSR), Pro-Asp-Ser-Gly-Arg (PDSGR), Ile-Lys-Val-Ala-Val (IKVAV) and Arg-Asn-Ile-Ala-Glu-Ile-Ile-Lys-Asp-Ala (RNIAEIIKDA).

27. The method according to claim 25, wherein the growth factor is selected from the group consisting of transforming growth factor-β (TGF), insulin-like growth factor (IGF), epidermal growth factor (EGF), nerve growth factor (NGF), vascular endothelial growth factor (VEGF), fibroblast growth factor (FGF), hepatocyte growth factor (HGF), platelet-derived growth factor (PDGF) and bone morphogenetic protein (BMP).
